(19) United States
(12) Patent Application Publication
(10) Pub. No.: US 2011/0262422 A1
(43) Pub. Date: Oct. 27, 2011

(54) ORALLY ADMINISTRABLE DOSAGE FORMS COMPRISING ANGIOGENIN AND USES THEREOF

(75) Inventors: Benjamin Cocks, Victoria (AU); Angus Tester, Victoria (AU); Peter Hobman, Victoria (AU); Matthew McDonagh, Victoria (AU)

(73) Assignees: MURRAY GOULBURN CO-OPERATIVE CO., LIMITED, BRUNSWICK, VICTORIA (AU); AGRICULTURE VICTORIA SERVICES PTY LTD., ATTWOOD, VICTORIA (AU)

(21) Appl. No.: 12/992,416

(22) PCT Filed: May 14, 2009
(86) PCT No.: PCT/AU09/00602

§ 371 Date: Mar. 24, 2011

(30) Foreign Application Priority Data
May 14, 2008 (AU) 2008902371

(51) Int. Cl. A61K 38/46; A61P 3/00; A61P 3/10; A61P 3/04; A61P 11/00; A61P 19/10; A61P 19/02; A61P 19/08; C12N 9/22; A61P 21/00

(52) U.S. Cl. 424/94.6; 435/199

(57) ABSTRACT

The invention provides a method of treatment of any disorder in which administration of angiogenin is beneficial wherein the angiogenin is administered orally. Particularly, the oral angiogenin does not require a carrier or excipient or for the protein to be encapsulated or subjected to any other mechanism to improve its oral bioavailability.

### FIELD

The present invention relates to oral preparations and particularly to oral preparations of therapeutic agents, particularly proteins and their use in methods of treatment.

### BACKGROUND

Oral administration of therapeutic agents is desirable because it is generally associated with optimal compliance by the patient with the treatment regimen, and permits greater flexibility of the dosing schedule, as well as avoiding the risks, inconvenience and expense associated with administration by injection. However, the ability to utilize the oral route is limited by the ability of the therapeutic agent to survive acid and enzymatic degradation in the oral cavity and digestive tract, and to pass across the epithelial cell layer into the systemic circulation.

This is particularly a problem with protein therapeutics, which are known to suffer from protease degradation in the gut and have problems crossing into the systemic circulation. Almost all pharmacological proteins are not orally available to a useful extent. In particular, hormones such as insulin, growth hormone, follicle-stimulating hormone or calcitonin, and cytokines such as interferon or interleukin, are known to have oral availabilities without special formulation well below 2%.

At such levels, the temporal and inter-individual variability in availability is typically high, rendering oral administration impractical, uneconomical or dangerous.

Protein drugs are of increasing importance in medical treatment. However, their use has been limited by the fact that the great majority of proteins have to be administered by injection. Although alternative routes of systemic administration have been suggested, such as the pulmonary, nasal or transdermal routes, hitherto these have been developed only for a limited range of agents and suffer from limitations in tolerability and in the amount of compound that can be delivered in a single dose.

Various attempts have been made to improve the bioavailability of pharmaceuticals. These include incorporation of penetration enhancers, such as salicylates, lipid-bile salt mixes, micelles, glycerides and acylcarnitines but these are found to cause toxicity problems on most occasions.

For protein therapeutics, attempts to improve oral bioavailability include mixing the protein or peptide with protease inhibitors such as aprotinin, soybean trypsin inhibitor and amastatin, to limit degradation of the administered therapeutic agent. Unfortunately these protease inhibitors are not selective and endogenous proteases are also inhibited by them with undesirable effects. Other attempts to provide oral formulations of proteins have utilized protective coatings such as enteric coatings, alone or together with chemical modification of the peptide by coupling of the protein or peptide to amphiphilic oligomers or polymers comprising for example a hydrophilic polyethylene glycol moiety and a lipophilic alkyl moiety. These techniques confer very limited success. Another approach is to add an excipient that loosens the tight junctions in the gastrointestinal tract, but this approach causes tolerability problems, because the compromised barrier may admit all molecules in the vicinity, including bacteria. Also calcium alginate-coated liposome formulations have been used for colonic delivery of peptides. However, so far such approaches have found only limited application.

It is an aim of a preferred embodiment of the present invention to provide an oral protein preparation capable of providing a biological effect.

All references, including any patents or patent applications, cited in this specification are hereby incorporated by reference. It will be clearly understood that, although a number of prior art publications are referred to herein, this reference does not constitute an admission that any of these documents forms part of the common general knowledge in the art.

### SUMMARY

A first aspect provides a method of treatment of any disorder in which administration of angiogenin is beneficial, wherein the angiogenin is administered orally.

It is particularly envisaged that the angiogenin will be administered orally, without the requirement for a carrier or excipient or for the protein to be encapsulated or subjected to any other mechanism to improve the protein's oral bioavailability.

Angiogenin is a 14 kDa, non-glycosylated polypeptide which is produced by several growing cell types including vascular endothelial cells, aortic smooth muscle cells, fibroblasts, and some tumours such as colon carcinomas, ovarian carcinomas, and breast cancers. Angiogenin has been isolated from a number of sources including normal human plasma, bovine plasma, bovine milk, and mouse, rabbit and pig sera and is also available recombinantly, for example as human or bovine recombinant angiogenin

Angiogenin has been implicated in a number of diseases and disorders. All treatments involving administration of angiogenin proposed by the prior art that may benefit from oral administration of angiogenin are within the scope of the present invention.

Whilst the prior art suggests oral delivery of angiogenin as one of a laundry list of routes of administration it has not previously been demonstrated that angiogenin is orally bioavailable. Accordingly a skilled person reading prior art suggestions that angiogenin be administered orally would have considered that carriers, excipients, encapsulation or other stabilising technology was necessary to allow the angiogenin to remain intact in the gut and yet be able to cross the gut wall into the bloodstream. The finding that angiogenin is orally bioavailable without the need for such manipulation is particularly unexpected and surprising.

Particularly the angiogenin may be administered orally as a food or food supplement, as a nutraceutical or as a pharmaceutical. The angiogenin may be human or bovine recombinant angiogenin or be extracted from any suitable source, for example milk or plasma.

Particularly contemplated is oral administration of a nutraceutical composition comprising an angiogenin enriched fraction from milk. Such fraction may be prepared using the method described in example 1 or by other suitable means.

A further aspect provides an oral dosage form of angiogenin. The angiogenin oral dosage form may also comprise follistatin or the oral dosage form of angiogenin may be provided in a kit which includes a dosage form of follistatin. The oral dosage form of angiogenin may take the forms of tablets, aqueous or oily suspensions, lozenges, troches, powders, granules, emulsions, capsules, syrups or elixirs.

### BRIEF DESCRIPTION OF FIGURES

FIG. 1 shows a one-dimensional SDS polyacrylamide gel of blood plasma samples taken at 2 and 3 hours following ingestion of low angiogenin dose (25 mg angiogenin, lanes 1 and 2 respectively), medium angiogenin dose (75 mg angiogenin, lanes 3 and 4 respectively) and high angiogenin dose (150 mg angiogenin, lanes 5 and 6 respectively). The level of angiogenin in the blood shows a clear increase from the low to the high dose across both 2 and 3 hour time points.

FIG. 2 shows angiogenin fed in the diet at 2.5 μg/g feed under ad libitum feeding conditions increases quadriceps weight in mice fed for 1 month and allowed to exercise freely on standard rodent running wheels.

FIG. 3 shows that angiogenin fed in the diet at 2.5 μg/g feed under ad libitum feeding conditions increases results in muscle fibre type cross sectional area changes in mice fed for 1 month and allowed to exercise freely on standard rodent running wheels. Group means for control animals are represented in white bars and group means for Angiogenin treated animals are represented in black bars. Standard deviations are given.

FIG. 4 shows that that angiogenin fed in the diet at 2.5 μg/g feed under ad libitum feeding conditions reduces the area of muscle necrosis in the quadriceps of MDX mice allowed to exercise freely on standard rodent running wheels.

### DETAILED DESCRIPTION

One aspect relates to the treatment of disorders. The terms “treating” and “treatment” as used herein refer to reduction in severity and/or frequency of symptoms, elimination of symptoms and/or underlying cause, prevention of the occurrence of symptoms (prophylaxis) and/or their underlying cause, and improvement or remediation of damage. Thus, for example, the present method of “treating” a disorder encompasses both prevention of the disorder in a predisposed individual and treatment of the disorder in a clinically symptomatic individual.

“Treating” as used herein covers any treatment of, or prevention of a condition in a vertebrate, a mammal, particularly a human, and includes: inhibiting the condition, i.e., arresting its development; or relieving or ameliorating the effects of the condition, i.e., cause regression of the effects of the condition.

“Prophylaxis” or “prophylactic” or “preventative” therapy as used herein includes preventing the condition from occurring or ameliorating the subsequent progression of the condition in a subject that may be predisposed to the condition, but has not yet been diagnosed as having it.

Persons of skilled in the art reading patent specifications suggesting that angiogenin can be administered orally would have appreciated that some method for improving the bioavailability of angiogenin was to be employed.

Known disorders and diseases in which administration of angiogenin has been suggested include those requiring angiogenesis. Oral treatment with angiogenin is therefore proposed for promoting development of a hemovascular network in a mammal, for example, to in collateral circulation following a heart attack, or to promote wound healing for example in joints or other locations. Angiogenin has been shown to possess a number of other activities, including a neuroprotective effect (Subramanian et al., (2007) Human Molecular Genetics vol. 17, no. 1 p130-149) and has been proposed to be useful in treating neurodegenerative diseases, such as ALS or motor neuron disease (see WO2006/054277). Angiogenin has also been proposed for inhibiting replication of RNA viruses in primary activated T cells and chronically infected cells and is proposed for treating RNA infection by Retroviridae, Cystoviridae, Birnaviridae, Reoviridae, Coronaviridae, Flaviviridae, Togaviridae, “Arterivirus”, Astroviridae, Caliciviridae, Picornaviridae, Potyviridae, Orthomyxoviridae, Filoviridae, Paramyxoviridae, Rhabdoviridae, Arenaviridae, and Bunyaviridae and is suggested for treating human immunodeficiency virus (see WO2004/106491). The inventors propose that such diseases or disorders may be treated by administering the angiogenin orally.

Our co-pending application proposes use of angiogenin in treating disorders involving follistatin, i.e. methods of treating a disorder associated with myostatin in an individual, methods of treating disorders where the interaction between follistatin and angiogenin can be used to improve function in tissues, methods of promoting muscle growth in an individual, methods of improving recovery of muscle from injury or use in an individual, methods of improving muscle strength in an individual, methods of improving exercise tolerance in an individual, methods of increasing the proportion of muscle in an individual, methods of decreasing fat in an individual and methods of decreasing an individual's fat to muscle ratio. All such methods can utilise angiogenin administered orally in accordance with the first aspect.

Other uses for orally administered angiogenin include methods to increase muscle mass, increase bone density, decrease muscle wasting, or for the treatment or prevention of conditions wherein the presence of myostatin causes or contributes to undesirable pathological effects or decrease of myostatin levels has a therapeutic benefit in mammals, preferably humans. In addition, angiogenin may be used to treat conditions where myostatin is not dysregulated, but improved follistatin mediated cell stimulation can be gained by addition of exogenous angiogenin.

Angiogenin can be used to reduce the severity of a pathologic condition, which is characterized, at least in part, by an abnormal amount, development or metabolic activity of muscle or adipose tissue in a subject. It can be administered to prevent, ameliorate or reduce the severity of a wasting disorder, such as cachexia, anorexia, AIDS wasting syndrome, muscular dystrophies, neuromuscular diseases, motor neuron diseases, diseases of the neuromuscular junction, and inflammatory myopathies.

The term “disorder associated with myostatin” refers to disorders of muscle, bone, or glucose homeostasis, and include disorders associated with abnormal myostatin.

A muscle is a tissue of the body that primarily functions as a source of power. There are three types of muscles in the body: a) skeletal muscle—striated muscle responsible for generating force that is transferred to the skeleton to enable movement, maintenance of posture and breathing; b) cardiac muscle—the heart muscle; and c) smooth muscle—the muscle that is in the walls of arteries and bowel. The method of the first aspect is particularly applicable to skeletal muscle but may have some effect on cardiac and or smooth muscle. Reference to skeletal muscle as used herein also includes interactions between bone, muscle and tendons and includes muscle fibres and joints.

Whilst angiogenin has previously been suggested to have an effect on cardiac muscle by virtue of its angiogenic activity and ability to provide increased blood flow to a muscle, this effect was restricted to oxidative muscles (type I and type IIa). The follistatin mediated effects of angiogenin on muscle as seen in the first aspect are distinct from those relating to angiogenesis as evidenced by all muscle fibres being affected.

The proposed uses of angiogenin on healthy individuals will be useful to athletes, both elite and amateur, body builders, those desirous of weight loss of enhanced physique and manual workers.

Since angiogenin is highly conserved in sequence and function across species, the method of the first aspect is applicable in non-human mammals or avian species [e.g. domestic animals (e.g., canine and feline), sports animals (e.g., equine), food-source animals (e.g., bovine, porcine and ovine), avian species (e.g., chicken, turkey, other game birds or poultry)] wherein the presence of myostatin causes or contributes to undesirable pathological effects or decrease of myostatin levels has a therapeutic benefit.

In a preferred embodiment of the method of the first aspect angiogenin is administered orally in the form of an angiogenin enriched extract from milk or plasma.

Particularly the orally administered angiogenin is prepared from cow's milk or a fraction thereof, for example using the process described in example 1. Such fraction has been found to provide angiogenin able to act systemically, without substantial degradation in the gut. Such fraction is able to be provided orally without employing carriers or other mechanisms to enhance the bioavailability of angiogenin.

The orally administered angiogenin may be or comprise recombinant angiogenin, particularly of human origin.

As referred to herein “oral delivery” or “oral administration” are intended to encompass any administration or delivery to the GI tract and includes administration directly to the oropharyngeal cavity, and administration via the mouth in which the actual absorption of the peptide or polypeptide takes place in the gastrointestinal tract, including the stomach, small intestine, or large intestine. Oral administration as used herein encompasses sublingual administration (administration by application under the tongue of the recipient, representing one form of administration via the oropharyngeal cavity) and buccal administration (administration of a dosage form between the teeth and the cheek of the recipient).

Oral delivery and oral administration may be used interchangeably herein.

Angiogenin is described as being “bioavailable” if it is present in the bloodstream of an individual to whom it has been administered in a functional form. “Functional form” means that the angiogenin is capable of having a therapeutic effect.

Whilst it is proposed that administration of angiogenin orally may act together with endogenous follistatin, oral angiogenin administered with follistatin (either simultaneously or sequentially) was shown by the inventors to have a more than additive effect compared to administration of follistatin alone or angiogenin alone.

Accordingly administration of oral angiogenin with follistatin is contemplated. The angiogenin and follistatin may both be provided orally, in the same or separate medicament, or the follistatin may be provided via another route, for example parenterally or via a transdermal patch.

The angiogenin may be provided in a pharmaceutical, veterinary or nutraceutical composition or as a food, particularly a functional food.

A pharmaceutical composition is one which is suitable for administration to humans. A veterinary composition is one that is suitable for administration to animals. Generally such compositions will contain purified angiogenin or at the very least all components of the composition will be verifiable.

The orally administered pharmaceutical or veterinary compositions used in the method of the first aspect may comprise one or more pharmaceutically acceptable carriers and optionally other therapeutic agents. Each carrier, diluent, adjuvant and/or excipient must be pharmaceutically “acceptable”.

By “pharmaceutically acceptable carrier” is meant a material which is not biologically or otherwise undesirable, i.e., the material may be administered to an individual along with the selected active agent without causing any undesirable biological effects or interacting in a deleterious manner with any of the other components of the pharmaceutical composition in which it is contained. Similarly, a “pharmaceutically acceptable” salt or ester of a novel compound as provided herein is a salt or ester which is not biologically or otherwise undesirable.

As used herein, a “pharmaceutical carrier” is a pharmaceutically acceptable solvent, suspending agent or vehicle for delivering the agent to the subject. The carrier may be liquid or solid and is selected with the planned manner of administration in mind. Each carrier must be pharmaceutically “acceptable” in the sense of being not biologically or otherwise undesirable i.e. the carrier may be administered to a subject along with the agent without causing any or a substantial adverse reaction.

Follistatin may be administered orally or parenterally. The term parenteral as used herein in relation to follistatin includes intravenous, intra arterial, intraperitoneal, intramuscular, subcutaneous, subconjunctival, intracavity, transdermal and subcutaneous injection, aerosol for administration to lungs or nasal cavity or administration by infusion by, for example, osmotic pump.

The angiogenin can be administered orally as tablets, aqueous or oily suspensions, lozenges, troches, powders, granules, emulsions, capsules, syrups or elixirs. Such composition may contain one or more agents elected from the group of sweetening agents, flavouring agents, colouring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate. The tablets may contain the agent in admixture with non-toxic pharmaceutically acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed.

Follistatin preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The compositions may also contain other active compounds providing supplemental, additional, or enhanced therapeutic functions. The pharmaceutical compositions may also be included in a container, pack, or dispenser together with instructions for administration.

Other therapeutically useful agents, such as growth factors (e.g., BMPs, TGF-P, FGF, IGF), cytokines (e.g., interleukins and CDFs), antibiotics, and any other therapeutic agent beneficial for the condition being treated may optionally be included in or administered simultaneously or sequentially with the angiogenin or angiogenin agonist.

Angiogenin or its agonists may also be presented for use in the form of veterinary compositions, which may be prepared, for example, by methods that are conventional in the art.

It is especially advantageous to formulate the veterinary or pharmaceutical compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms are dictated by and directly dependent on the unique characteristics of the active compound and the particular therapeutic effect to be achieved, and the limitations inherent in the art of compounding such an active compound for the treatment of individuals.

Pharmaceutical or veterinary compositions comprising angiogenin or an agonist thereof are to be administered in therapeutically effective amounts. As used herein, an “effective amount” of angiogenin is a dosage which is sufficient to reduce the activity of myostatin to achieve a desired biological outcome. The desired biological outcome may be any therapeutic benefit including an increase in muscle mass, an increase in muscle strength, improved metabolism, decreased adiposity, or improved glucose homeostasis. Such improvements may be measured by a variety of methods including those that measure lean and fat body mass (such as duel ray scanning analysis), muscle strength, serum lipids, serum leptin, serum glucose, glycated hemoglobin, glucose tolerance, and improvement in the secondary complications of diabetes.

Generally, a therapeutically effective amount may vary with the subject's age, condition, and sex, as well as the severity of the medical condition in the subject. The dosage may be determined by a physician and adjusted, as necessary, to suit observed effects of the treatment. Appropriate dosages for administering angiogenin or its agonists may range from 5 mg to 100 mg, from 15 mg to 85 mg, from 30 mg to 70 mg, or from 4 mg to 60 mg. The compositions can be administered in one dose, or at intervals such as once daily, once weekly, and once monthly.

Dosage schedules can be adjusted depending on the half life of angiogenin or its agonist, or the severity of the patient's condition.

Generally, the compositions are administered as a bolus dose, to maximize the circulating levels of angiogenin for the greatest length of time after the dose. Continuous infusion may also be used after the bolus dose.

It is also contemplated that the methods utilise a nutraceutical composition to provide the angiogenin. A nutraceutical composition for use in the methods is provided.

The term “nutraceutical” as used herein refers to an edible product isolated or purified from food, in this case from a milk product, which is demonstrated to have a physiological benefit or to provide protection or attenuation of an acute or chronic disease or injury when orally administered. The nutraceutical may thus be presented in the form of a dietary preparation or supplement, either alone or admixed with edible foods or drinks.

A functional food is a foodstuff to which a composition has been added to give that food a physiological benefit or to provide protection or attenuation of an acute or chronic disease or injury when orally administered.

The nutraceutical composition or functional food may be in the form of a soluble powder, a liquid or a ready-to-drink formulation. Alternatively, the nutritional composition may be in solid form as a food; for example in the form of a ready-to-eat bar or breakfast cereal. Various flavours, fibres, sweeteners, and other additives may also be present.

The nutraceutical preferably has acceptable sensory properties (such as acceptable smell, taste and palatability), and may further comprise vitamins and/or minerals selected from at least one of vitamins A, B1, B2, B3, B5, B6, B11, B12, biotin, C, D, E, H and K and calcium, magnesium, potassium, zinc and iron.

The nutraceutical composition may be produced as is conventional; for example, the composition may be prepared by blending together the protein and other additives. If used, an emulsifier may be included in the blend. Additional vitamins and minerals may be added at this point but are usually added later to avoid thermal degradation.

If it is desired to produce a powdered nutraceutical composition, the protein may be admixed with additional components in powdered form. The powder should have a moisture content of less than about 5% by weight. Water, preferably water which has been subjected to reverse osmosis, may then be mixed in to form a liquid mixture.

If the nutraceutical composition is to be provided in a ready to consume liquid form, it may be heated in order to reduce the bacterial load. If it is desired to produce a liquid nutraceutical composition, the liquid mixture is preferably aseptically filled into suitable containers. Aseptic filling of the containers may be carried out using techniques commonly available in the art. Suitable apparatus for carrying out aseptic filling of this nature is commercially available.

Preferably the nutraceutical composition also comprises one or more pharmaceutically acceptable carriers, diluents or excipients. Nutraceutical compositions may comprise buffers such as neutral buffered saline, phosphate buffered saline and the like; carbohydrates such as glucose, mannose, sucrose or dextrans; mannitol; proteins; polypeptides or amino acids such as glycine; antioxidants; chelating agents such as EDTA; adjuvants and preservatives.

The nutraceutical may be an infant formula, particularly a humanised milk formula for administration to infants. Such an infant formula may find utility in treating failure to thrive or premature or low birth weight babies. It may also be administered to infants or children to improve cognitive function.

The angiogenin used in the method of the first aspect may be from any source. It may be natural, synthetic or recombinant in origin. Recombinant angiogenin can be based on the angiogenin sequence from any species, including humans, cows, sheep, mouse, etc. Recombinant human angiogenin is available from R & D Systems.

Angiogenin is known to be present in normal human plasma, bovine plasma, bovine milk, bovine plasma and mouse, rabbit and pig sera. The DNA and protein sequences of at least human angiogenin are available and recombinant human angiogenin is available commercially from Abnova Corporation (Taiwan) for small scale applications.

In one embodiment the angiogenin is prepared from plasma or milk from livestock animals as readily available sources of angiogenin on a commercial scale.

The milk may be obtained from any lactating animal, e.g. ruminants such as cows, sheep, buffalos, goats, and deer, non-ruminants including primates such as a human, and monogastrics such as pigs. In a preferred embodiment the angiogenin is extracted from cow's milk. The animal from which angiogenin is produced may be a transgenic animal designed to over-express human or bovine angiogenin in its milk.

The inventors of the present application have shown that in bovine milk, angiogenin is present in the highest or most concentrated amount (up to 12 mg/litre) within the first 1 to 14 days of lactation. Following this, the concentration falls to a base level of approximately 1 to 2 mg/litre. Therefore it is preferred that cow's milk which obtained within the first 14 days of lactation as a source of angiogenin for use in the methods of the first to eleventh aspects. Given the residual angiogenin levels in cow's milk from later lactation, it may still be used a source for the method of the first aspect.

The angiogenin used in the method of the first aspect may be isolated or purified. Purified or isolated angiogenin is substantially free of at least one agent or compound with which it is naturally associated. For instance, an isolated protein is substantially free of at least some cellular material or contaminating protein from the cell or tissue source from which it is derived. The phrase “substantially free of cellular material” refers to preparations where the'angiogenin is at least 50 to 59% (w/w) pure, at least 60 to 69% (w/w) pure, at least 70 to 79% (w/w) pure, at least 80-89% (w/w) pure, at least 90-95% pure, or at least 96%, 97%, 98%, 99% or 100% (w/w) pure.

Recombinant angiogenin preparations in bacteria may be used as a source of angiogenin and may be provided in the form of protein aggregates.

As bovine milk is a natural product that has been in food chain for hundreds of years, the angiogenin used as a nutraceutical need not be totally pure. However, to reduce the amount of composition to be administered it is preferred that the angiogenin is concentrated significantly with respect to its concentration in milk. Preferably the angiogenin is administered in at a concentration of at least 10 times its concentration in milk and more preferably 20, 30, 40, or 50 times its concentration in milk.

When provided as a food the angiogenin can take the form of a food supplement, a nutritional formulation or an infant formula.

Persons skilled in the art will appreciate that variants of bovine angiogenin exist in nature and can be manufactured.

Persons skilled in the art will appreciate that the angiogenin used may be modified to improve storage stability, bioactivity, circulating half life, or for any other purpose using methods available in the art. For example it may be desirable to introduce modification to improve storage stability. However, as angiogenin is particularly resistant to degradation such modification may not be essential.

The first aspect refers to agonists of angiogenin. An agonist is a compound that is capable of directly or indirectly having an effect through the receptor activated by angiogenin. Preferably angiogenin agonists act through the angiogenin receptor and preferably bind the receptor. Persons skilled in the art will appreciate how to design agonists of angiogenin. Suitable agonists include angiogenin agonist antibodies and mimetic compounds.

Angiogenin, its agonists and variants may be used in the manufacture of a medicament for administering orally in the method of the first aspect, particularly in the form of an angiogenin enriched extract from milk or plasma or in the form of recombinant angiogenin

Particularly the orally administered angiogenin is prepared from cow's milk or a fraction thereof, for example using the process described in example 1. Such fraction has been found to provide angiogenin able to act systemically, without substantial degradation in the gut. Such fraction is able to be provided orally without employing carriers or other mechanisms to enhance the bioavailability of angiogenin.

Angiogenin is anticipated to interact with endogenous follistatin (if recombinant angiogenin is used) or the enriched angiogenin extract may also contain follistatin. Administration of angiogenin plus follistatin (either simultaneously or sequentially in any order) is shown herein to have a more than additive effect and accordingly each of the methods of treatment contemplate administration of follistatin with angiogenin. It is particularly important to co-administer (either simultaneously or sequentially) follistatin with angiogenin in situations where an individual is follistatin deficient. As follistatin levels decrease with age, co-administration of follistatin with angiogenin is particularly contemplated when treating the elderly.

In a co-administration regime, angiogenin may be administered orally and follistatin administered orally or otherwise.

There may be occasion when it is not desirable to administer angiogenin. As the inventors have found that angiogenin is orally available in milk, they also provide milk having a reduce concentration of angiogenin for use on such occasions.

In one embodiment the milk having a reduced concentration of angiogenin has 40%, 50%, 60%, 70%, 80%, 90% less angiogenin than bovine whole or skim milk or is substantially angiogenin free.

Methods for making milk having reduced angiogenin will be apparent to persons skilled in the art. For example the milk flow through from a cation exchange column or an immunoaffinity column comprising anti-angiogenin antibodies will have reduced angiogenin compared to the milk sample applied to the column. Electodialysis would also be expected to provide milk depleted for angiogenin (and also for lactoferrin and lactoperoxidase). A suitable method for making milk with a reduced angiogenin content are provided in the examples.

In the description of the first aspect and in the claims which follow, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used herein, the singular forms “a”, “an”, and “the” include the corresponding plural reference unless the context clearly dictates otherwise. Where a range of values is expressed, it will be clearly understood that this range encompasses the upper and lower limits of the range, and all values in between these limits.

Unless defined otherwise, all technical and scientific terms used herein have the same meaning as commonly understood by one of ordinary skill in the art to which this invention belongs. Although any materials and

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

The invention will now be described in detail by way of reference only to the following non-limiting examples and drawings.

### Example 1

### Process for the Preparation of an Angiogenin-Enriched Fraction from Skim Milk

A 10 cm deep column was packed with SP Sepharose Big Beads (GE Healthcare) such that the total bed volume of the column was 29.7 litres. To the column a flow of skimmed cow's milk was applied at a linear flow rate of 331 cm/h (34 litres of skimmed milk per litre of resin per hour) for 2 hours until the volume of skimmed milk applied was 68 times the volume of the resin packed into the column.

The milk remaining in the column was removed by adding 2.5 column volumes (CV) of water at a linear flow rate of 147 cm/h (15 litres of buffer per litre of resin per hour), or 0.25 CV/min, for 10 min.

The angiogenin-depleted lactoperoxidase fraction was eluted from the column with 2.5 CV of a buffer containing sodium ions equivalent to 2.0% (0.34M) NaCl, at pH 6.5, by flowing the cation buffer solution at a linear flow rate of 75 cm/h (7.5 litres of cation buffer solution per litre of resin per hour), or 0.125 CV/min, for 20 min. The first 0.5 litres of cation buffer solution per litre of resin was discarded to drain and the next 2.5 litres of cation buffer solution per litre of resin was collected as the angiogenin-depleted lactoperoxidase fraction (including 0.5 litres of cation buffer solution per litre of resin overlapping the application time of the next buffer, i.e. breakthrough time).

The angiogenin-enriched fraction was then eluted from the column with 2.5 CV of a buffer containing sodium ions equivalent to 2.5% w/v (0.43 M) NaCl, at pH 6.5, by flowing the cation buffer solution at a linear flow rate of 75 cm/h (7.5 litres of cation buffer solution per litre of resin per hour), or 0.125 CV/min, for 20 min. The first 0.5 litres of cation buffer solution per litre of resin was discarded to drain and the next 2.5 litres of cation buffer solution per litre of resin was collected as the angiogenin-enriched fraction (including 0.5 litres of cation buffer solution per litre of resin overlapping the application time of the next buffer).

Finally, the lactoferrin fraction (again angiogenin depleted) is eluted from the column with 2.5 CV of a buffer containing sodium ions equivalent to 8.75% w/v (1.5 M) NaCl, at pH 6.5, by flowing the cation buffer solution at a linear flow rate of 75 cm/h (7.5 litres of cation buffer solution per litre of resin per hour), or 0.125 CV/min, for 20 min. The first 0.5 litres of cation buffer solution per litre of resin was discarded to drain and the next 2.5 litres of cation buffer solution per litre of resin was collected as the lactoferrin fraction.

The angiogenin-enriched fraction that was collected was ultrafiltrated (NMWCO 5 kDa) to concentrate and reduce the salt content. The resultant concentrate was freeze-dried and stored at room temperature for subsequent use.

The angiogenin-enriched fraction was analysed for angiogenin content by SDS-PAGE and the fraction was found to contain 57% (protein basis) of a low molecular weight (14 kDa) protein which was confirmed to be angiogenin by MALDI-TOF/TOF MS (results not shown).

Persons skilled in the art would appreciate that angiogenin from other sources or purified by other means could be used in the method of the first aspect. The above example is merely to show how the actual source of angiogenin used in the following experiments was made and is in no way intended to be limiting.

Whilst it may be considered that the angiogenin enriched fraction may contain additional bioactive components which are having an effect, the comparable amount of angiogenin as available in skim milk (concentration 2%) had comparable activity in the examples shown to the angiogenin enriched fraction (data not shown).

### Example 2

### Detection of Bovine Angiogenin in Human Blood

To analyse the ability of bovine angiogenin to cross into the blood stream following ingestion, duplicate human subjects consumed a dose of the angiogenin enriched milk fraction described in example 1 that was equivalent of either 25 mg, 75 mg or 150 mg of angiogenin. The angiogenin dose was made up in 100 ml of commercially available flavoured milk immediately prior to ingestion. Prior to ingestion, a time 0 blood sample was taken. Blood samples were then taken at 2 and 3 hours after ingestion.

The presence of angiogenin is confirmed by SDS-PAGE performed according to the method of Laemmli, 1970 (Nature 227(5259): 680-685), and staining with SYPRO Ruby according to manufacturer's instructions. For example, an aliquot of the protein faction is denatured in SDS PAGE buffer (usually containing 2% sodium dodecyl sulphate, 10% glycerol, 50 mM Tris HCL (pH 6.8), 2 mM EDTA, 140 mM β-mercaptoethanol and 0.01% bromophenol blue) by heating at 95° C. for 5 minutes. Denatured samples are loaded onto SDS PAGE gels (can be sourced from a provider; for example Invitrogen Novex precast Tris HCl gels). The gel is placed into the SDS PAGE apparatus and running buffer (Tris base 3.03 g/L, glycine 14.4 g/L and SDS 1.0 g/L) is placed in the bottom and top wells of the apparatus. Electrophoresis is conducted at 200V for 1 hour. Following electrophoresis, proteins are fixed within the gel using fixing/destain solution (10% methanol and 7% acetic acid) prior to staining for at least 1 hr with Sypro Ruby (Invitrogen). Gels are destained for at least 1 hr in fixing/destain solution prior to imaging on a gel scanning system. Abundance of angiogenin is determined by analysis of the distinct band at approximately 15 kDa.

The presence of angiogenin can also be confirmed by immunoaffinity detection following SDS PAGE by western blotting with an anti-bovine angiogenin antibody. Biological samples containing bovine angiogenin would be dissolved in 1× NuPAGE LDS sample buffer (Invitrogen) with or without reducing agent (2-mercaptoethanol). The samples are then heated at 95° C. for 5-10 minutes prior to SDS PAGE electrophoresis as described above. Following electrophoresis, the gel is then placed in Towbin buffer (25 mM Tris, 192 M glycine, 1% SDS) for 10 minutes. The Western transfer procedure would use a commercially available transfer device such as the iBlot™ (Invitrogen) and transfer would be performed according to the manufacturers' instructions. The iBlot™ Anode Stack (Bottom) is removed from its packaging and placed on the bottom of the iBlot™ gel transfer device. The pre-run gel is then positioned on the nitrocellulose membrane (iBlot™ membrane; Invitrogen). One sheet of iBlot™ Filter Paper which had been soaked in deionized water is then placed on top of the pre-run gel. The iBlot™ Cathode Stack (Top) is then placed on top of the pre-soaked filter paper with the copper electrode side facing up. The iBlot™ Disposable Sponge was then placed on the inner side of the lid to ensure contact with the iBlot™ transfer stack. Voltage is applied across the apparatus for 7 minutes at 25V. At the end of the transfer, the nitrocellulose membrane is placed in blocking solution (TBST containing 5% skim milk powder) for 1 hour. The membrane is then transferred into the primary antibody solution. This solution contains a suitable dilution of an anti-bovine monoclonal such as clone number 1B14D4 (Property of the Department of Biochemistry, Chungbuk National University, Cheongju, Chungbuk, Korea) or polyclonal antibody in TBST (10 mM Tris HCl, pH 7.5, 150 mM NaCl, 0.05% Tween20) containing 5% BSA. The nitrocellulose membrane is incubated in the primary antibody solution overnight at 4° C. with gentle shaking. The nitrocellulose membrane is then washed 4 times for 5 minutes each at room temperature in TEST with gentle shaking. The nitrocellulose membrane is then placed in the secondary antibody solution which contains a labelled antibody for detecting the primary antibody. In the case of the anti-bovine angiogenin monoclonal antibody 1B14d4, an IRDye 800CW Goat Anti-Mouse IgG secondary (Licor) diluted 1 in 15,000 in TBST containing 5% skim milk powder may be used. In this case, the nitrocellulose membrane is incubated in the secondary antibody solution in the dark for 45 minutes at room temperature. The nitrocellulose membrane is then washed 4 times for 5 minutes each at room temperature in TBST with gentle shaking in the dark. The nitrocellulose membrane was then washed with TBS (10 mM Tris HCl, pH 7.5, 150 mM NaCl) and dried between two sheets of filter paper (Whatman). The nitrocellulose membrane is then scanned at the appropriate wavelength using an infrared imager (Licor Odyssey). The specific abundance of angiogenin is determined by analysis of the distinct band at approximately 15 kDa.

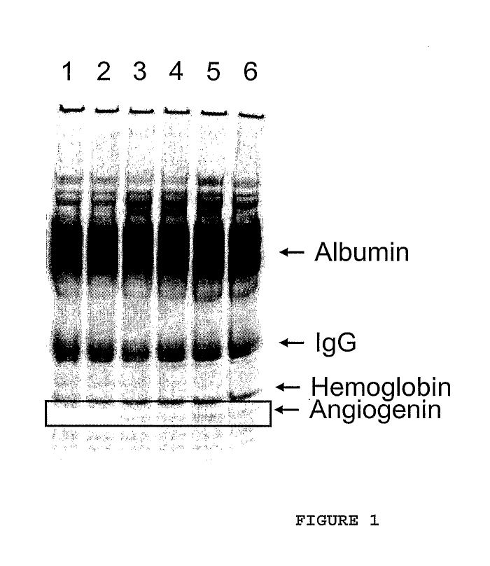

As shown in FIG. 1 the level of angiogenin in the blood shows a clear increase from the low to the high dose across both 2 and 3 hour time points, showing that angiogenin is orally available.

### Example 3

### In Vivo Animal Studies

To analyse the bioavailability of angiogenin in normal mice, animal studies were undertaken. All work was approved by the University of Western Australia animal ethics committee.

Mice were fed 2 diets during each trial; a control diet and a diet containing bovine angiogenin (bAngiogenin) enriched fraction made according to example 1 at 2.5 μg/g mouse weight. These studies were carried out on adult (8 wks of age) male normal (C57) and dystrophic (mdx) mice with n=8 for each mouse strain per diet for each experiment.

Normal mice were subjected to a one month dietary period with ad libitum access to feed and voluntary exercise; for voluntary exercise a metal mouse wheel is placed inside the cage and the distance run by individual mice is recorded by a bicycle pedometer attached to the wheel. MDX mice were subjected to the same one month dietary period. In separate experiments, mdx mice were given the voluntary exercise treatment described above or were given no voluntary exercise wheel.

### Experimental Analysis:

During the experiments body weight, amount of food eaten and muscle strength (grip strength test) were all measured twice weekly. At the conclusion of each experiment the mice were sacrificed by halothane anaesthesia and cervical dislocation.

Experimental mice will then be used for the following analysis to determine any changes in phenotype as a result of treatments on dystrophic and normal muscle.

1) Body Composition analysis: Half of each skinned mouse carcase was analysed for body composition. In addition individual leg muscles including the quadriceps (quad), tibialis anterior (TA) and gastrocnemius muscles were dissected and weighed as well as the abdominal fat pads and heart were recorded to determine gross phenotypic changes induced by the diets.

2) Histological analysis: Skeletal muscle and heart samples were collected and prepared for both frozen and paraffin histology. Histological analysis will be performed on the following muscles, quad, TA and diaphragm. Haematoxylin and Eosin, Sudan Black and various immunohistological stains will be performed on these muscles. Skeletal myofibre necrosis, myofibre hypertrophy and fat content of muscles will be determined.

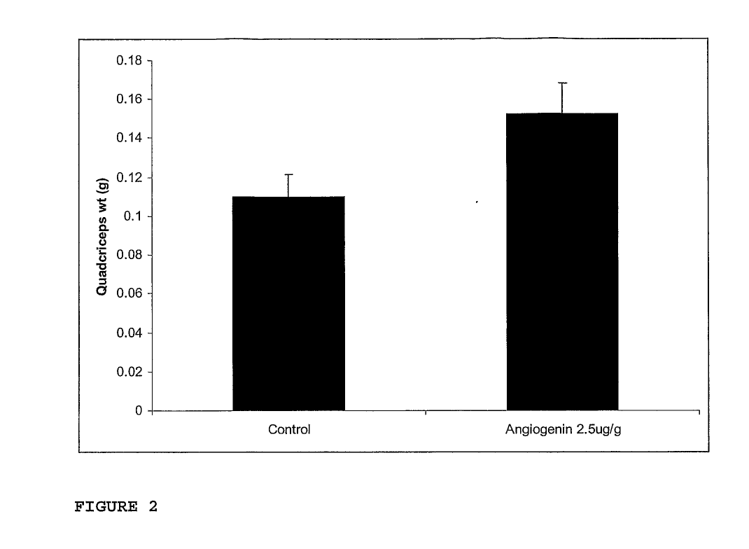

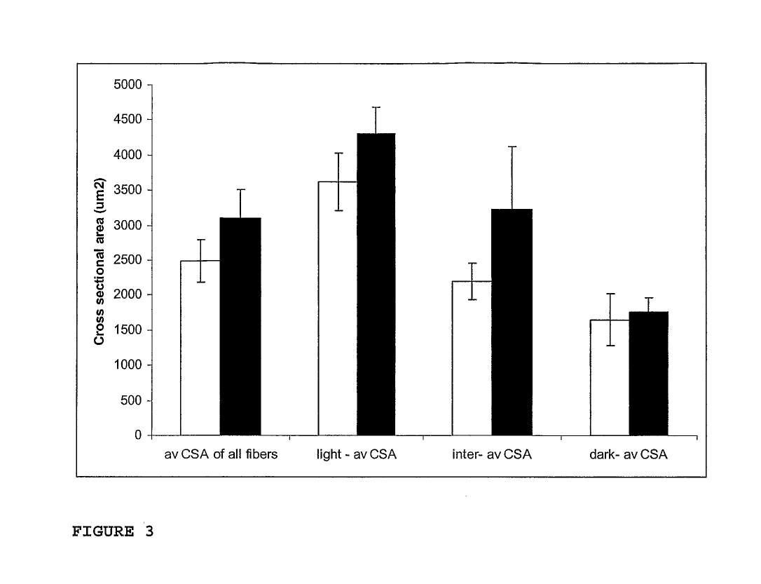

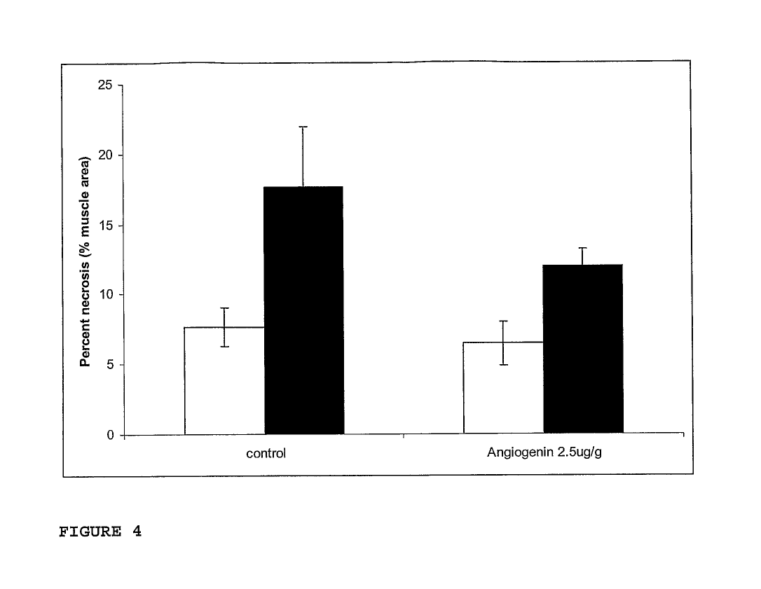

Results from the in vivo experiments in normal mice are shown in FIGS. 2 and 3. It is clear that the diet supplemented with angiogenin enriched fraction at 2.5 μg/g induces muscle gain (FIG. 2) of up to 50% compared to the control group. Increase in muscle mass was accommodated by increased cross sectional area of most muscle fibre types except for the population of small dark fibres corresponding to slow-twitch oxidative fibres (FIG. 3). This demonstrates bioavailability of angiogenin when taken orally, showing that angiogenin crosses the gut and is active at the tissue level. When fed to mdx mice, angiogenin reduced the proportion of the muscle that was necrotic when mice were allowed access to voluntary exercise (FIG. 4). This demonstrates that angiogenin is bioavailable following ingestion in mdx mice and is capable of inhibiting the effects of exercise on muscle breakdown in mdx mice. This means that the orally administered angiogenin is capable of having its therapeutic effect despite being administered orally.

### Example 4

### Reduction of Angiogenin in Skimmed Milk

A column with a depth of 100 mm and a volume of 30 L was filled with SP Big Bead cation exchange resin (GE Healthcare). The column was prepared by rinsing with 180 L (6 column volumes) 1.0M sodium chloride at 1,300 L/h and then 180 L (6 column volumes) water at 1,800 L/h (milk and all solutions were pH 6.5±1). Skim milk was applied to the resin at a rate of 1,300 L/h until 2,100 L (70 column volumes) were applied. Samples of skim milk were collected immediately before and immediately after the column. The milk collected immediately after the column is angiogenin-reduced milk. The skim milk and angiogenin-reduced milk were frozen in stainless steel trays, freeze-dried (temperature, 50° C.; time, 48 h; vacuum, 1 mBar) and then sealed in an air-tight foil pouch until analysis could occur.

Angiogenin was pre-concentrated by low pressure cation exchange chromatography and then measured by cation exchange HPLC. Milk powder (20 g) was dissolved in 400 mL water by stirring with a magnetic stirring bar for 30 min. Hydrochloric acid (2 M) was added until the milk solution reached pH 4.6. Precipitated casein was removed by sequential filtration through a Whatman #113 and then Whatman #1 filter papers and the clarified whey was collected. The whey was adjusted to pH 6.2±0.1 by the addition of 30% sodium hydroxide.

SP Big Bead cation exchange resin (35 g wet weight) was added to the whey and stirred for 1 h. The whey and resin were poured into a disposable 10 mL column in many batches until all of the resin accumulated in the column. The column was rinsed with distilled water and then eluted with 4M sodium chloride solution. The eluate from the column was monitored at 280 nm to ensure that all the protein was collected. The volume of eluate collected was 45 g. The eluate (200 μL) was diluted to 1000 μL by adding distilled water (necessary to allow proper analysis of the salt-rich solution by HPLC) and 100 μL was analysed by cation exchange HPLC. The area of angiogenin present in skim milk was 8,461 mAU×s and the area of angiogenin in angiogenin-reduced milk was 893 mAU×s, which represents an 89.44% reduction in the amount of angiogenin present.

1-11. (canceled)

12. A method of treating of disorder in which administration of angiogenin is beneficial, the method comprising administering angiogenin orally.

13. The method of claim 12 in which the angiogenin is administered orally, without the requirement for a carrier or excipient or for the protein to be encapsulated or subjected to any other mechanism to improve the protein's oral bioavailability.

14. The method of claim 12 in which the disorder is selected from the group consisting of metabolic diseases, insulin-dependent (type 1) diabetes mellitus, noninsulin-dependent (type 2) diabetes mellitus, hyperglycemia, impaired glucose tolerance, metabolic syndrome, syndrome X, insulin resistance induced by trauma, adipose tissue disorders, obesity, muscle and neuromuscular disorders, sarcopenia, cachexia, muscular dystrophy, severe or benign X-linked muscular dystrophy, limb-girdle dystrophy, facioscapulohumeral dystrophy, myotinic dystrophy, distal muscular dystrophy, progressive dystrophic ophthalmoplegia, oculopharyngeal dystrophy, Duchenne's muscular dystrophy, and Fakuyama-type congenital muscular dystophy; amyotrophic lateral sclerosis (ALS); muscle atrophy; organ atrophy; frailty; carpal tunnel syndrome; congestive obstructive pulmonary disease; congenital myopathy; myotonia congenital; familial periodic paralysis; paroxysmal myoglobinuria; myasthenia gravis; Eaton-Lambert syndrome; secondary myasthenia; denervation atrophy; paroxymal muscle atrophy; sarcopenia, cachexia, muscle wasting syndromes, osteoporosis; especially in the elderly and/or postmenopausal women; glucocorticoid-induced osteoporosis; osteopenia; osteoarthritis; osteoporosis-related fractures; traumatic or chronic injury to muscle tissue, low bone mass due to chronic glucocorticoid therapy, premature gonadal failure, androgen suppression, vitamin D deficiency, secondary hyperparathyroidism, nutritional deficiencies, anorexia nervosa, disorders in which promoting angiogenesis would be beneficial, and wound healing.

15. The method of claim 12 in which the angiogenin comprises recombinant angiogenin.

16. The method of claim 12 in which the angiogenin is derived from bovine milk.

17. The method of claim 12 further comprising administering follistatin.

18. The method of claim 17 in which the follistatin is administered parenterally.

19. The method of claim 17 in which angiogenin and follistatin are co-administered.

20. A composition comprising angiogenin in an oral dosage form.

21. The composition of claim 20 further comprising follistatin.

22. A kit comprising angiogenin in an oral dosage form and follistatin.
